(19) United States
(12) Patent Application Publication
(10) Pub. No.: US 2020/0000366 A1
(43) Pub. Date: Jan. 2, 2020

(54) IN-BODY BACKSCATTER COMMUNICATION AND LOCALIZATION

(71) Applicant: Massachusetts Institute of Technology, Cambridge, MA (US)

(72) Inventors: Dina Katabi, Boston, MA (US); Omid Salehi-Abari, Whitby (CA); Deepak Vasisht, Cambridge, MA (US); Guo Zhang, Cambridge, MA (US)

(21) Appl. No.: 16/456,406

(22) Filed: Jun. 28, 2019

Related U.S. Application Data
(60) Provisional application No. 62/692,310, filed on Jun. 29, 2018.

(51) Int. Cl. A61B 5/06; H01Q 1/27; H04B 17/391; H01Q 1/24; G06K 19/07; A61B 5/00; A61B 5/1459; A61B 5/1473

(52) U.S. Cl. CPC A61B 5/061; H01Q 1/273; H04B 17/391; H01Q 1/248; A61B 2560/0214; A61B 5/7225; A61B 5/1459; A61B 5/1473; G06K 19/0723

(57) ABSTRACT

A backscatter approach is particularly customized for deep tissue devices, which do not require active signal transmission for localization of or data communication from the devices. The design overcomes interference from the body surface, and localizes the in-body backscatter devices even though the signal travels along non-straight paths. Data communication for the in-body device is also available using the approach.

### CROSS-REFERENCE TO RELATED APPLICATIONS

This application claims the benefit of U.S. Provisional Application No. 62/692,310, filed Jun. 29, 2018, which is incorporated herein by reference.

### PRIOR DISCLOSURES BY INVENTOR

Deepak Vasisht, Guo Zhang, Omid Abari, Hsiao-Ming Lu, Jacob Flanz, and Dina Katabi. “In-body backscatter communication and localization.” In Proceedings of the 2018 Conference of the ACM Special Interest Group on Data Communication (SIGCOM), pp. 132-146. ACM, August, 2018.

### BACKGROUND

This application related to communication and/or localization using radio frequency backscatter from an in-body device.

The medical industry is looking at a wide array of in-body devices that include pacemakers that communicate their data over the wireless channel, smart pills that image the gastrointestinal tract, and microscale robots that access organs through the bloodstream. Today, such deep tissue systems communicate by generating their own radio signal, a process that consumes a lot of energy. For instance, in wireless capsule endoscopes, RF consumes 4 to 10 times more power than the sensors [35]. As a result, these capsules use large batteries that occupy about 40-50% of the space of the capsule [3, 6]. Reducing the power requirement for RF transmissions can reduce the size of the capsules making them more easy to swallow. It can also improve completion likelihood. Past work has found that 16.5% of the times, capsule endoscopes fail to completely visualize the small bowel primarily due to limited battery life [26].

Similarly, interest in deep tissue localization is on the rise. Localization of deep tissue sensors like capsule endoscope can enable physicians to isolate the parts of the GI tract with abnormalities, adapt video frame rates based on location, and deposit biomarkers at specific locations [27, 31]. The localization requirements for such capsules are on the order of a few centimeters [27].

Past work has tried to tackle these problems along multiple axes. Researchers have considered wireless power transfer—i.e., charging an implant using RF signals [21, 1]. These systems typically operate in the midfield where the RF transmitter is either directly in touch with the body or within a few centimeters from it.

The literature also has few proposals for in-body localization. One line of research uses magnetic field analysis [27, 2, 14]. The advantage of using the magnetic field is that its properties do not change much between air and human tissues. The disadvantage however is that the magnetic field power decays with a factor d⁶ as it travels through air [8]. Hence, the magnetic receiver (the receiving coil) has to be in touch with the body surface or within a few centimeters. Further, magnetic implants can be problematic. They can be painful if the person is exposed to a strong magnetic field as in the case of MRI [17]. They can also affect MRI images making it difficult to detect a tumor in the area near the implant [17]. A result, this form of localization is not widely used. Doctors also use X-ray or sonar for localization. These methods are expensive. Further, continuous tracking of an implant requires excessive x-ray exposure which increases cancer risk [9]. Finally, the use of ultrasound for in-body localization requires direct contact with the human skin, making it infeasible for several medical applications. For instance, presence of metallic equipment close to the human body can be an hindrance for administering X-ray/proton beams used for radiation therapy in cancer treatments [24, 10].

Some proposals for in-body RF-based localization have used the received signal strength (RSS) [34, 33]. Those systems use an array of receive antennas and either assume the implant to be closest to the receive antenna with the highest power or use path loss models to estimate location [31]. Analysis of the error bounds on RSS in-body localization has reported lower bounds of 4 to 6 cm [34] even when using up to 50 receive antennas. Past work has also tried to adapt indoor localization based on time-of-flight (ToF) or angle of arrival (AoA) for the domain [32, 22, 5]. Unfortunately, these systems are based purely on simulation, lack any empirical results, and most of them ignore signal deflection.

There is also a rich literature related to backscatter communication and localization in-air [4, 36, 15, 19]. However, in general, this work does not address problems that stem from RF propagation in deep tissues, such as signal deflection and body surface interference. The design in [36, 15, 18] proposes shifting the frequency of the backscatter signal to avoid WiFi interference. Also, some past work on harmonic RADARs and RFID-based localization ([28, 7, 13, 12]) has used non-linearity to mix two frequencies and weed out unwanted reflections from the environment.

### REFERENCES

[1] A. Abid, Jonathan M. O'Brien, T. Bensel, C. Cleveland, L. Booth, B. R. Smith, R. Langer, and G. Traverso. Wireless power transfer to millimeter-sized gastrointestinal electronics validated in a swine model. Nature Scientific Reports, 2017.
[2] S. M. Aziz, M. Grcic, and T. Vaithianathan. A Real-Time Tracking System for an Endoscopic Capsule using Multiple Magnetic Sensors. Springer Berlin Heidelberg, 2008.
[3] M. R. Basar, F. Malek, K. M. Juni, M. S. Idris, and M. I. M. Saleh. Ingestible wireless capsule technology: A review of development and future indication. International Journal of Antennas and Propagation, 2012.
[4] D. Bharadia, K. R. Joshi, M. Kotaru, and S. Katti. BackFi: High Throughput WiFi Backscatter. ACM SIGCOMM, 2015.
[5] R. Chandra, A. J. Johansson, and F. Tufvesson. Localization of an rf source inside the human body for wireless capsule endoscopy. BodyNets, 2013.
[6] X. Chen, X. Zhang, L. Zhang, X. Li, N. Qi, H. Jiang, and Z. Wang. A wireless capsule endoscope system with low-power controlling and processing asic. IEEE Transactions on Biomedical Circuits and Systems, 2009.
[7] B. G. Colpitts and G. Boiteau. Harmonic radar transceiver design: miniature tags for insect tracking. IEEE Transactions on Antennas and Propagation, 2004.
[8] W. contributors. Magnetic dipole—wikipedia, the free encyclopedia, 2017. https://en.wikipedia.org/w/index.php?title=Magnetic_dipole&oldid=811519977.
[9] A. B. de GonzÃ [symbol] lez and S. Darby. Risk of cancer from diagnostic x-rays: estimates for the uk and 14 other countries. The Lancet, 2004.
[10] I. Dietlicher, M. Casiraghi, C. Ares, A. Bolsi, D. Weber, A. Lomax, and F. Albertini. Experimental measurement with an anthropomorphic phantom of the proton dose distribution in the presence of metal implants. PTCOG, 2014.
[11] K. R. Foster and J. Jaeger. Rfid inside. IEEE Spectrum, 2007.
[12] H. Gomes and N. B. Carvalho. Rfid for location proposes based on the intermodulation distortion. Sensors & Transducers, 2009.
[13] H. C. Gomes and N. B. Carvalho. The use of intermodulation distortion for the design of passive rfid. In 2007 European Radar Conference, 2007.
[14] C. Hu, M. Q. Meng, and M. Mandal. Efficient magnetic localization and orientation technique for capsule endoscopy. In 2005 IEEE/RSJ International Conference on Intelligent Robots and Systems, 2005.
[15] P. Hu, P. Zhang, M. Rostami, and D. Ganesan. Braidio: An Integrated Active-Passive Radio for Mobile Devices with Asymmetric Energy Budgets. ACM SIGCOMM, 2016.
[16] Institute of Applied Physics. Dielectric Properties of Body Tissues. http://niremf.ifac.cnr. ithissprop/htmlclie/htmlclie.php.
[17] E. Kanal, A. J. Barkovich, C. Bell, J. P. Borgstede, W. G. B. Jr, J. W. Froelich, J. R. Gimbel, J. W. Gosbee, E. Kuhni-Kaminski, P. A. Larson, J. W. L. Jr, J. Nyenhuis, D. J. Schaefer, E. A. Sebek, J. Weinreb, B. L. Wilkoff, T. O. Woods, L. Lucey, and D. Hernandez. Acr guidance document on mr safe practices: 2013. Journal Of Magnetic Resonance Imaging, 2013.
[18] B. Kellogg, V. Talla, S. Gollakota, and J. R. Smith. Passive wi-fi: Bringing low power to wi-fi transmissions. USENIX NSDI, 2016.
[19] V. Liu, A. Parks, V. Talla, S. Gollakota, D. Wetherall, and J. R. Smith. Ambient Backscatter: Wireless Communication out of Thin Air. ACM SIGCOMM, 2013.
[20] R. Lodato, V. Lopresto, R. Pinto, and G. Marrocco. Numerical and experimental characterization of through-the-body uhf-rfid links for passive tags implanted into human limbs. IEEE Transactions on Antennas and Propagation, 2014.
[21] A. Ma and A. S. Y. Poon. Midfield wireless power transfer for bioelectronics. IEEE Circuits and Systems Magazine, 2015.
[22] D. Manteuffel and M. Grimm. Localization of a functional capsule for wireless neuro-endoscopy. In 2012 IEEE Topical Conference on Biomedical Wireless Technologies, Networks, and Sensing Systems (BioWireleSS), 2012.
[23] K. Michael. Rfid/nfc implants for bitcoin transactions. IEEE Consumer Electronics Magazine, 2016.
[24] C. Oancea, K. Shipulin, G. Mytsin, A. Molokanov, D. Niculae, I. Ambrožvá, and M. Davídková. Effect of titanium dental implants on proton therapy delivered for head tumors: experimental validation using an anthropomorphic head phantom. Journal of Instrumentation, 2017.
[25] S. J. Orfanidis. Electromagnetic waves and antennas. Rutgers University New Brunswick, N.J., 2002.
[26] G. Ou, N. Shahidi, C. Galorport, O. Takach, T. Lee, and R. Enns. Effect of longer battery life on small bowel capsule endoscopy. World Journal of Gastroenterology, 2015.
[27] D. M. Pham and S. M. Aziz. A real-time localization system for an endoscopic capsule using magnetic sensors. IEEE Sensors, 2014.
[28] K. Rasilainen, J. Ilvonen, A. Lehtovuori, J. M. Hannula, and V. Viikari. On design and evaluation of harmonic transponders. IEEE Transactions on Antennas and Propagation, 2015.
[29] Skyworks. SMS7630 Series. http://www.skyworksinc.com/Product/511/SMS7630_Series?IsProduct=true.
[30] Taoglas. PC 30 Antenna. http://www.taoglas.com/product/pc30-2g3g-cellular-fr4-pcb-antenna-mmcxmra-2/.
[31] I. Umay, B. Fidan, and B. Barshan. Localization and tracking of implantable biomedical sensors. IEEE Sensors, 2017.
[32] I. Umay, B. Fidan, and M. R. YÃ¼ce. Endoscopic capsule localization with unknown signal propagation coefficients. In 2015 International Conference on Advanced Robotics (ICAR), 2015.
[33] Y. Wang, R. Fu, Y. Ye, U. Khan, and K. Pahlavan. Performance bounds for rf positioning of endoscopy camera capsules. In 2011 IEEE Topical Conference on Biomedical Wireless Technologies, Networks, and Sensing Systems, 2011.
[34] Y. Ye and K. Pahlavan. Accuracy bounds for and rss and toa based rf localization in capsule endoscopy. 2011.
[35] M. R. Yuce and T. Dissanayake. Easy-to-swallow wireless telemetry. IEEE Microwave Magazine, 2012.
[36] P. Zhang, D. Bharadia, K. Joshi, and S. Katti. HitchHike: Practical Backscatter Using Commodity WiFi. ACM SenSys, 2016.

### SUMMARY

In-body devices are becoming increasingly common in the medical industry. Some examples of in-body devices include pacemakers, smart pills (e.g., endoscopic pills), and microscale robots. Many in-body devices communicate with devices outside of the body by emitting radio frequency (RF) signals. For example, smart pills emit RF signals to communicate sensor data to receivers outside of a patient's body. To overcome the high attenuation associated with transmitting RF signals through multiple centimeters of tissue, many conventional in-body devices are active devices that transmit using battery power. One disadvantage of conventional, battery powered in-body devices is that their batteries often increase the size of the devices (e.g., the battery occupies more than 40% of the device).

For in-body devices that operate in deep tissue (i.e., centimeters below the skin), it is often the case that localization of those devices is required. For example, a gastroenterologist may need to know where an endoscopic pill is located in the gastrointestinal tract when a particular image is transmitted from the pill.

Aspects described herein are related to the use of backscatter communication techniques for communication with and/or localization of deep tissue in-body devices. Very generally, an external device is able to communicate with and localize a deep-tissue, passive backscatter device in the presence of skin reflections and through a number of layers of tissue, each being associated with a different propagation speed of radio frequency signals.

In some examples, the external device transmits RF signals at two or more different frequencies from one or more transmit antennas. The transmitted signals traverse one or more layers of tissue of a subject and reach an in-body device, causing the in-body device to emit a backscatter signal. The in-body device is a passive backscatter device that includes a non-linear circuit which causes the emitted backscatter signal to include a known mixture of the frequencies transmitted from the transmit antennas.

The backscatter signal and skin reflections of the transmitted signals are received at a number of receive antennas. Prior to digitization, the skin reflections (which are generally much stronger than the backscatter signal) are filtered out, leaving only the backscatter signals.

The backscatter signals received at the receive antennas are processed to localize the passive backscatter device in the subject's tissue. The processing accounts for the effects of different propagation speeds of radio frequency signals in the one or more layers of tissue.

In some examples, the internal device (e.g., “implant”, “smart pill”) has the ability to harvest power from received radio-frequency signals, while leveraging backscatter to communicate at zero power and save its harvested energy for its sensing tasks.

In some examples, the approach separates skin reflections from the signal from in-body implants, and solves unique localization challenges (like signal deflections, change of wavelength in-body) that do not exist in in-air localization.

In one exemplary application, aspects are used in cancer radiation therapy for accurate localization of tumors. For example, if, during cancer radiation treatment, the tumor being treated moves, there is a potential for significantly damaging tissues around the tumor. This is particularly a problem with proton therapy where the main benefit is the ability to accurately radiate at a particular depth with minimal exposure to the surrounding tissues. However, the tumor could move during the process causing the radiation beam to fall on the wrong tissue. For example, a breast cancer tumor may move with a patient's breathing, or a prostate cancer tumor may move due to bowel movements. Aspects described herein use wireless radio signals to track tumor movements during radiation by embedding a small marker (e.g., a passive backscatter device) in the body to assist in pinpoint and tracking tumor locations. In some examples, if localization of the passive backscatter device indicates that the tumor under treatment has moved beyond some threshold, the radiation stops until the tumor returns to its prior location. Alternatively, the beam could be steered to track the change in tumor position.

In a general aspect, a localization method includes receiving, at each antenna of a number of antennas, an emitted signal from a passive device located in a subject's body, each antenna of the number of antennas providing a respective received signal of a number of received signals, wherein the emitted signal includes a first set of frequency components and is caused by subjecting the passive device to a transmitted signal including a second set of frequency components not included in the first set of frequency components and processing the number of received signals to determine a location of the passive device, the processing being based at least in part on the effects of different propagation speeds of the emitted signal in one or more layers of tissue through which the emitted signal passes to reach the number of antennas.

Aspects may include one or more of the following features.

The passive device may include circuitry for forming the first set of frequency components from the second set of frequency components. The circuitry may include non-linear circuitry. The circuitry may include a diode. The transmitted signal may include a first frequency component with a first frequency and a second frequency component with a second frequency. The first set of frequency components includes a mixture of the first frequency and the second frequency. The method may include processing the received signals to remove components at frequencies of the second set of frequency components. Determining the location of the passive device may include determining a first set of distances between the number of antennas and the passive device and determining a second set of distances by processing the first set of distances according to the effects of the different propagation speeds of radio frequency signals in the one or more layers of tissue through which the emitted signal passes to reach the number of antennas.

The effects of the different propagation speeds of radio frequency signals in the one or more layers of tissue through which the emitted signal passes to reach the number of antennas may include refraction and changes in wavelength. The location of the passive device may be determined, at least in part, using a model of human tissue. The model may define the one or more layers of tissue as including an oil-based tissue layer and a water-based tissue layer.

In another aspect, in general, an in-body device includes an antenna and a non-linear circuit coupled to the antenna. The combination of the antenna and the non-linear circuit is configured to, when excited by a radio frequency signal including signal components at a first set of two of more frequencies, emit a radio frequency signal including signal components at a second set of frequencies that is distinct from the first set of frequencies.

Aspect can include one or more of the following features.

The non-linear circuit comprises a diode. For example, the non-linear circuit comprises a Schottky detector diode. The non-linear circuit may be a passive circuit.

The device further comprises circuitry configured to modulate the emitted radio frequency signal.

The circuitry configured to module the emitted radio frequency signal comprises a modulating element coupled to the antenna and the non-linear circuit. For example, the modulating element comprises a transistor.

The circuitry configured to modulate the emitted radio frequency signal comprises transmission circuitry for receiving data and outputting a control signal from controlling the modulating element.

The device comprises a sensor configured to acquire sensor data in the body and wherein the device is configured to modulate the emitted radio frequency signal according to the acquired sensor data. For example, the sensor comprises at least one of a camera, and electrical sensor, and a biochemical sensor.

The device comprising an energy harvesting component coupled to the antenna configured to convert received radio-frequency energy to power for operating circuitry of the in-body device.

In another general aspect, a kit includes an external device configured to perform any or all of the steps described above. Aspects may include a passive device for introduction into a subject, the passive device configured to perform any or all of the steps described above.

### DESCRIPTION OF DRAWINGS

FIG. 1 is a schematic diagram showing use of a sensing system.

FIG. 2 is a block diagram of the sensing system.

FIG. 3 is a diagram illustrating signal propagation paths.

FIG. 4 is a flowchart of operation of the sensing system.

FIG. 5 is a flowchart of a localization approach.

FIG. 6 is a diagram of signal paths from the backscatter module.

FIG. 7 is a diagram of signal paths illustrating a distance computation.

FIG. 8 is a block diagram of the backscatter module.

### DETAILED DESCRIPTION

Overview

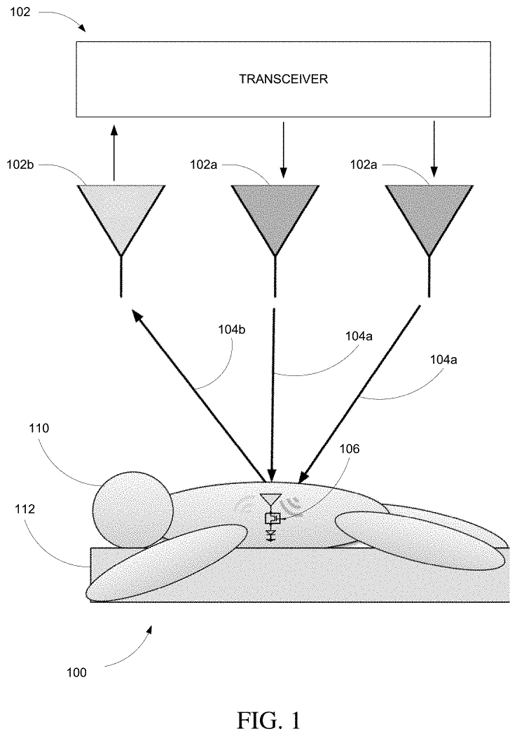

Referring to FIG. 1, a system 100 for localization and/or communication uses radio frequency signals to locate a device in a body, to receive data from the device, or both. Very generally, the system 100 includes a transceiver 102 that emits radio frequency signals via one or more transmit antennas 102a. These signals propagate through air and then through body tissue. Components of the signals reaching a backscatter module 106 in the body cause the module to emit other radio frequency signals, which then propagate through the body and through the air back to one or more receive antennas 102b at the transceiver. These signals that are emitted from the backscatter device 106 include components at different frequencies than those emitted from the transceiver. The difference in transmitted and backscatter frequences mitigates interference from direct or reflected paths of the originally emitted signals from the transceiver. In at least some embodiments, the backscatter device is passive in the sense that the signals emitted from the backscatter device are a result of the interaction of the signals emitted from the transceiver with the device, rather than being a result of an active transmission (e.g., radio frequency power amplification, modulation, etc.) of the emitted signals. However it should be recognized that the backscatter module may have active components, for example, digital or analog circuitry (e.g., for sensing or data processing) powered by energy harvested from the received signals emitted from the transceiver.

In some embodiments, two patch antennas 102a may be used for transmissions and three patch antennas 102b (not all shown in FIG. 1) for reception. Very generally, the multiple paths between pairs for transmit antennas 102a and receive antennas 102b are used for localization. However, in embodiments where localization is not required, a single receive antenna 102b is sufficient for communication.

As discussed in more detail below, the transceiver emits radio signals at two (or more) different frequencies, and the backscatter device causes a combination of the signals at those two frequencies to be combined in a non-linear manner causing the signals emitted from the backscatter device to include components at other frequences than the two frequencies emitted from the transceiver, generally at harmonic combinations of those emitted frequencies. To avoid mixing of the transmitted frequencies in the transmission circuit causing harmonic combinations of the two frequencies, separate transmit chains may be used for each of the transmitted frequencies. One choice for the transmit frequencies is 830 MHz (f₁) and 870 MHz (f₂), with two harmonics received from the backscatter device being at 910 MHz (2f₂−f₁) and 1700 MHz (f₁+f₂). Of course other transmitted frequencies and harmonics may be used. In at least some embodiments, instead of transmitting a constant frequency signal while localizing or communicating with the backscatter device, the transmitted signals sweep through 8 MHz of bandwidth. As discussed further below, such sweeping may be used to improve localization accuracy.

In some embodiments, the antennas may be placed from 50 cm to 2 m away from the subject, and may be connected to Universal Software Radio Peripheral (USRP) software radios. The backscatter module 106 is located within a body 110 (e.g., surgically emplanted, introduced into the digestive tract, etc.). The body is permeable with respect to the signals produced by the transmitting antennas 102a of the transceiver 102. In some configurations, the body 110 is placed on a surface 112. In some embodiments, the setup can be placed below or above a bed or on the side, relative to body 110. The in-body module may be a small unit that can be attached to standard in-body devices that need to communicate data or be localized. While it is common to perform medical procedures while the patient is lying on a bed, the operation does not necessarily require the body 110 to be in a particular position.

Further referring to FIG. 1, each transmitting antenna 102a produces a corresponding transmitting signal 104a. The transmitting signal 104a reaches the patient's body 110, where it is partically reflected back into the air, and partially passed into the body and then received by the backscatter module 106. As introduced above, the backscatter module 106 then in turn emits a signal based in part on the signal it just received. The transceiver 102 receives a combined signal 104b, which includes reflected components (e.g., from the skin surface, or possibly from object in the vicinity) as well as the harmonic signals produced by the backscatter module 106. The combined signal is received by the transceiver 102 via the set of receiving antennas 102b.

As discussed further below, one embodiment of the backscatter module 106 used a diode connected to an antenna of the module. In operation, given a received signal from the multiple transmit antennas 104a (i.e., the received signal having multiple frequency components), the diode present in the backscatter module 106 causes the mixing of the frequencies of the components in the received signal thereby creating second and third order harmonic frequencies. In order to communicate from the backscatter device, a switch modulates (e.g., on-off) the connection of the antenna and the diode, thereby modulating the backscattered signals. The transceiver then demodulates the backscatter signal using corresponding demodulation techniques.

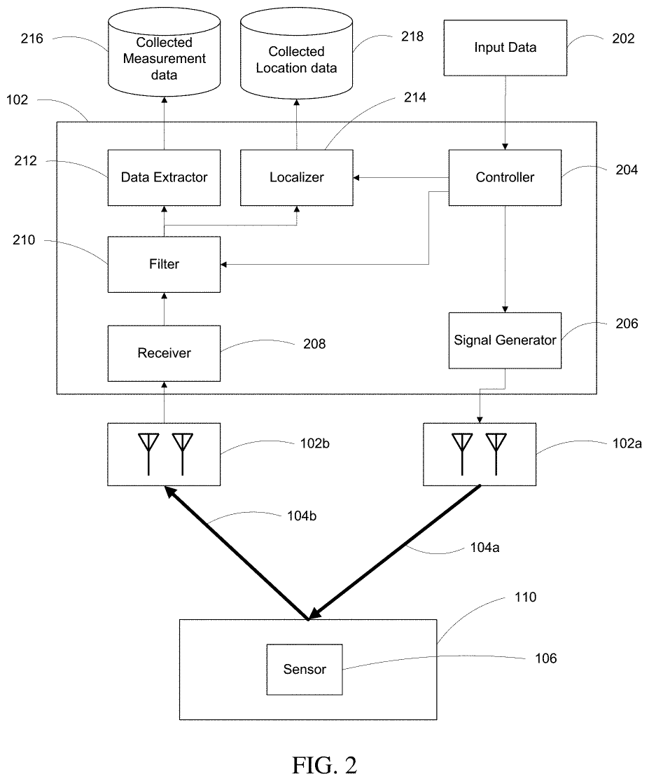

Referring to FIG. 2, a block diagram shows internal components or modules of the system 100. The transceiver 102 is shown to further include a controller 204 which receives input data or commands 202 and provides a control signal to each of a signal generator 206, a filter 210, and a localizer 214. The characteristics of the aforementioned control signal produced by the controller 204 are based on input data 202 provided to the controller 204 in operation of the system 100. The signal generator 206 receives a control signal from the controller 206 and causes the set of transmitting antennas 102a to emit the transmit signals 104a based on that received control signal. As introduced above, in some embodiments, the transmitting signal 104a consists of two frequencies (f₁ and f₂), with one frequency emitted from each of the transmit antennas 102a. while the signal measured at the receiver after being backscattered is at f₁+f₂, 2f₁+f₂, and other frequency combinations.

Further referring to FIG. 2, the combined reflected and backscatter signal 104b is acquired by the set of receiving antennas 102b and passed to a receiver 208, for example, that amplifies the received signals. The receiver 208 passes the received signals a filter 210 (e.g., multiple analog circuitry filters, one for each antenna) to process the data in preparation for being sent to a data extractor 212 and a localizer 214. Generally, the filter attenuates signal components at the originally transmitted frequencies (f₁ and f₂), and passes the harmonic frequencies that have been selected for localization and data communication (e.g., f₁+f₂ and 2f₁+f₂). In embodiments in which the transmit frequencies scan over a range of frequencies, the stop and pass bands are sufficient to accommodate the scanned frequency ranges. Further regarding the filter 210, in addition to the signal from the receiver 208, the filter also receives as input a signal from the controller 204. In some embodiments, the controller 204 may be configured to supply the filter with a set of parameters from which it will base its filtering process (e.g., the selected pass bands). Once the data has been filtered, it is now in a format conducive to use by the data extractor 212 and the localizer 214. The data extractor 212 digitizes the signal extracts any data field from the filtered signal and stores it in a Collected Measurement Data database 216. In some embodiments, the data present in the data field may be information gathered by the backscatter module 106 which, in further embodiments, may relate to measurements taken regarding the patient 110. The localizer 214 receives the filtered data from the filter 210 and processes location data from the filtered data by putting it in a Collected Location Data database 218. This location data is used to characterize the position of the backscatter module 106 with respect to the transceiver 102.

In some embodiments, the controller 204 provides an input signal to the Localizer 214 that includes information to parameterize the collected location data. In further embodiments, this input signal from the controller 204 to the localizer 214 contains information about the signal created by the signal generator 206, which is compared against the measured result supplied by the filter 210.

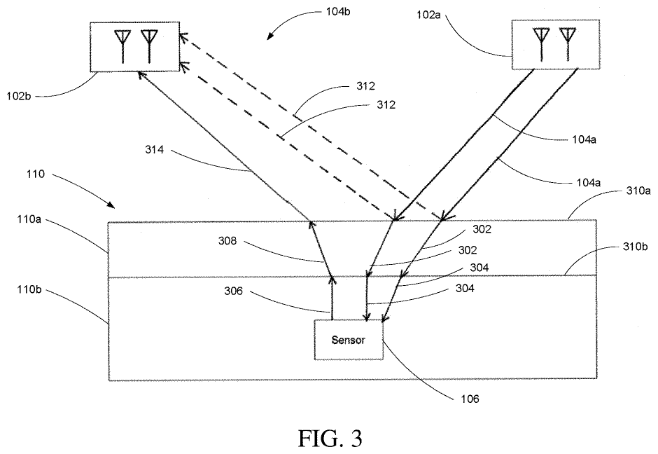

Referring to FIG. 3, detailed view of the operation of backscatter module 106 inside the patient 110 is shown from the point of view of signal propagation paths. In FIG. 3 the transmitting antennas 102a emit corresponding transmitting signals 104a (i.e., the two signals at frequencies f₁ and f₂, respectively). The receiving antennas 102b are in turn shown to be receiving a combined signal 104b, which includes reflected signals 312 at the transmit frequencies, as well as a backscatter signal 314. That is, each receive antenna 102b receives a combination of the reflected signals 312 and the backscattered signal 314, which each receive antenna 102b receiving a different combination (e.g., with different phase combinations) based on the physical paths followed by the signal from the transmit antennas 102b to that receive antenna.

In general, the body 110 may be considered to be made up of multiple layers of body material, each with different radio propagation characteristics. In FIG. 3, the body 110 is shown to be made up of a first body layer 110a and a second body layer 110b, where the first body layer 110a is on the surface of the body disposed directly on top of the second layer 110b, and the backscatter device 106 is located within the second layer. A first surface 310a is between the air and the first layer 110a, and a second surface (or interface) 310b is between the first layer 110a and the second layer 110b. This figure is an illustration, and it is not required that there be such multiple layers, or there be only two layers, for the described techniques to be applicable. When a signal 104a reaches the first surface 310a of the first layer 110a, part of the signal energy is reflected as a signal 312 at the same frequency as was transmitted, and part of the signal passes into the body as signal 302. Generally, because of the difference in propagation properties (e.g., propagation speed) the signal 104a is refracted forming the internal signal 302 (i.e., a singly-refacted signal), and therefore the signal does not follow a straight path from the transmit antenna 104a inside the body. The singly-refracted transmitting signal 302 then passes through the second surface 310b into the second body layer 110b as a doubly-refacted signal 304. Because of difference in the propagation characteristics in the second layer, the direction of travel of the doubly-refracted signal 304 is again different than the singly-refracted signal 302.

Further referring to FIG. 3, the doubly-refracted signal 304 reaches the backscatter module 106, which is situated inside the second body layer 110b. In embodiments that communicate data from (or to) the backscatter module, the backscatter module 106 is energized by the doubly-refracted signal, powering electronics allowing it to receive information in the signal 304 or to modulate a backscatter signal. Whether or not the backscatter module is energized, the interaction of the received signals 304 with non-linear circuitry in the module (e.g., a diode-based circuit), the module emits a backscatter signal 306, which passes through the second surface 310b and into the first body layer 110a from the second body layer 110b, refracted, becoming a singly-refracted backscatter signal 308. When the singly-refracted signal 308 passes through the first surface 310a into open air from the first body layer 110a, it is refracted once more into a doubly-refracted backscatter signal 314, which ultimately arrives as a component of the received signal 104b at a receive antenna 102b. The combined signal 104b is shown to include both the doubly-refracted backscatter signal 314 and reflected transmitting signals 312. The reflected transmitting signal 312 is characterized as the portion of the transmitting signal 104a which is reflected by the patient 110 and never reaches the backscatter module 106 as a result. The combined reflected signal 104b is then received by the receiving antennas 102b, and is processed in a manner consistent with that which was previously described in the operation with reference to FIG. 2.

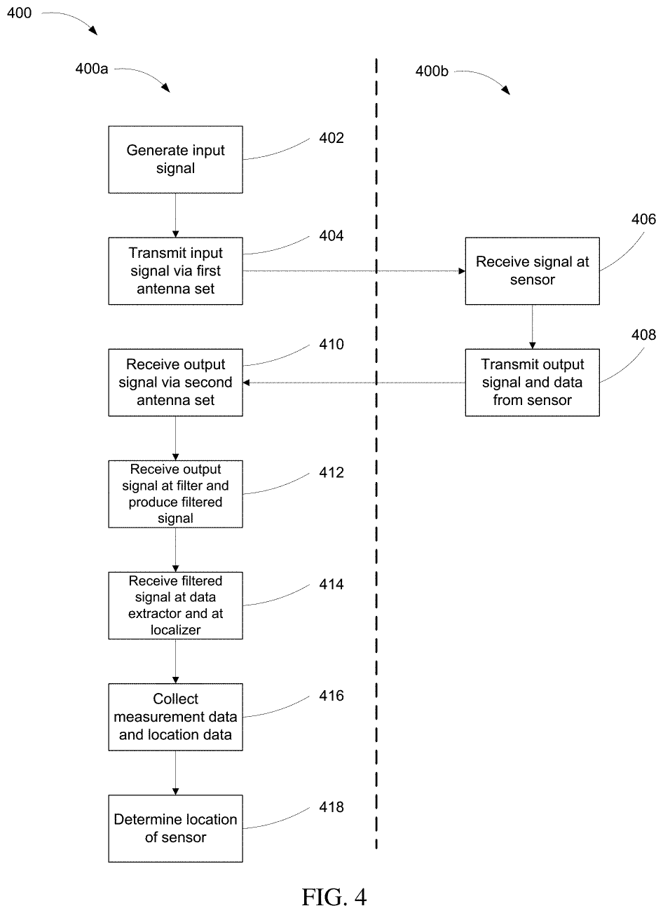

Referring to FIG. 4, a flowchart 400 describing the operation of the system illustrates a first operation flow 400a and a second operation flow 400b, where the operations in the first operation flow 400a are performed by the transceiver 102 (see FIG. 2), and the operations in the second operation flow 400b are performed entirely by the backscatter module 106. The operation described by flowchart 400 begins with the generation of an input signal (e.g., a frequency scan around f₁ or f₂) (402). This input signal is then transmitted via antennas 102a of first antenna set (404). In relation to the system illustrated in FIG. 2, the first antenna set described in step (404) are the transmitting antennas 102a. The signal is then received by a sensor (406), which subsequently transmits output data (408). As steps 406 and 408 are both part of the second operation flow 400b, in operation of the system they are performed entirely by the backscatter module. Within the context of the system illustrated in FIG. 2, this means operations 406 and 408 are performed by backscatter module 106.

Further referring to FIG. 4, the output signal transmitted as part of step 408 is received via antennas 102b of the second antenna set (410), the operation of the system returning back to the first operation flow 400a. The received signal is then received at a filter which then produces a filtered signal (412). Next, the filtered signal is sent to a data extractor and a localizer (414) which then allow for the collection of the measurement data and the location data (416). Finally, using the collected measurement and location data, the operation determines the location of the sensor (418).

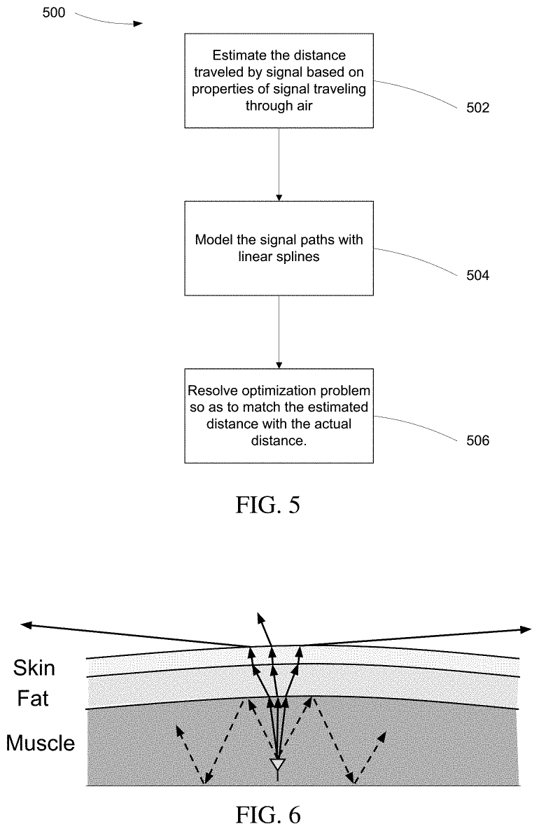

Referring to FIG. 5, a flowchart 500 describing the algorithm by which the localizer (as described in FIG. 2) localizes the backscatter module via received signals is shown. First, the localizer estimates the distance traveled by a received signal assuming the signal traveled entirely through air (502). In practice, this characterization is referred to as the effective-in-air distance. In some embodiments, the transmitter has two transmit antennas that transmit two signals f₁ and f₂, and the receiver includes a number of receive antennas. In this embodiment, let d₁ and d₂ be the effective distances from the two transmitters to the backscatter device, and dᵣ the effective distance from the backscatter device to receiver r. The transmitters are transmitting frequencies f₁ and f₂, while the receivers receive the non-linear mixing of these two signals at frequencies f₁+f₂, 2f₁−f₂, and other linear combinations.

In operation, in-air effective distance does not translate into physical distances directly. Furthermore, since the signals do not travel in a straight line or at constant speed, distance cannot simply be intersected from different transmitters and receivers to get an accurate location. In some embodiments, signal propagation is modeled as linear splines (instead of a straight line). This allows for propagation in each layer to be represented as linear while allowing for a change of direction across layers. In further embodiments, modeling the individual segments of the splines as functions of the latent variables in the model facilitates leveraging the observed effective in-air distances to estimate the latent variables. By doing so, this optimization can accurately estimate the position of the device by modeling the spline structure.

Further referring FIG. 5, the localizer then proceeds to model the signal path with linear splines (piecewise linear segments) (504). Here, the length of each segment refers to the stretch of the path in a particular material (for example, the stretch of the path traveling through fat would be represented by a different segment than the stretch of the path traveling through muscle). The localizer then solves an optimization problem that maps effective-in-air distances to the correct splines that match the actual paths traveled by the signal (506).

Principles of Operation

Principles of operation of one or more embodiments are presented below, without necessarily limited the approach to be based or or to be consistent with these principles.

RF Signals in the Body

The manner in which RF signals behave as they propagate in biomaterial (e.g., fat, muscles) have implications for in-body backscatter, and therefore in the details of the design of the localization aspects of the system. From the perspective of electromagnetic (EM) waves, each material is characterized by two parameters: relative electrical permittivity, ϵᵣ and relative magnetic permeability, μᵣ. These are complex numbers that capture how the electrical and magnetic fields in an EM wave interact with the material. Both ϵᵣ and μᵣ are 1 for air and vacuum. For biological tissues, the relative magnetic permeability μᵣ can be approximated as 1, so we set μᵣ=1 in embodiments of the localization approach. However, ϵᵣ has high variability depending on the tissue type and frequency of transmission. For example, for frequencies around 1 GHz (commonly used by in-body implants), the value of ϵᵣ in muscle is 55−18j [16].

The value of ϵᵣ is very important because it changes the speed of light and other electromagnetic waves (EM) in a material. Specifically, the speed of light in a biomaterial (e.g., muscle, fat, skin) is given by:

$$v = \frac{c}{\sqrt{{ϵ}_{r}}} ,$$

where c is the speed of light in vacuum and (to a good approximation) air. The change in the speed of the EM wave has important implications.

For a signal at frequency f, traveling in free space from a transmitter to a receiver separated by distance d, the wireless channel h(f,d) is given by

$$h ( f , d ) = \frac{A}{d} {e}^{- j 2 π f \frac{d}{c}} \qquad ( 1 )$$

where A is the attenuation constant that depends on the antenna beam patterns and c is the speed of light in vacuum.

For biomaterial, incorporating EM wave speed change in equation (1) gives us the wireless channel, h_{M}(f,d):

$${h}_{M} ( f , d ) = \frac{A}{d} {e}^{- j 2 π f \frac{d \sqrt{{ϵ}_{r}}}{c}} \qquad ( 2 )$$

To understand the impact of ϵᵣ on wave propagation, let us write \sqrt{ϵᵣ}=α−βj, where α and β are positive real numbers. The channel equation can then be updated as:

$${h}_{M} ( f , d ) = \frac{A}{d} {e}^{- j 2 π f \frac{d ( α - β j )}{c}} = \frac{A}{d} {e}^{- 2 π f \frac{d α}{c}} {e}^{- 2 π f \frac{d β}{c}} \qquad ( 3 )$$

Note that the term

$${e}^{- 2 π f \frac{d β}{c}}$$

causes exponential loss in magnitude of the signal during propagation. The higher the value of β, the higher the loss. This is in addition to the propagation attenuation experienced by the signal in free space, given by

$$\frac{A}{d} .$$

Also, muscle tissues experience significant additional loss in comparison with in-air signals.

Based on the considerations outlined above, in-body RF signals should use relatively low frequencies to avoid the drastic power loss occurring at higher frequencies. In fact, it is a common practice to use frequencies about 1 GHz, which are small enough to have a relatively low loss, but also large enough to enable relatively small electronics and antennas. Also, for backscatter signals which have to traverse the body twice, they lose more than 20 dB just to get 5 cm deep.

The electrical permittivity of a material further affects the efficiency of in-body antennas. As an antenna is placed in-body, its radiation efficiency decreases and its inherent losses increase as a function of ϵᵣ. For muscle tissues, these effects incur another 10-20 dB of loss depending on the antenna design.

Consider again Eq. 3. Note that the signal phase changes much faster in biomaterial than in air. Specifically, the phase changes a times faster in biomaterial than in air. This is because the wavelength is a times smaller. This property is useful for RF-based localization algorithms that leverage phase changes to measure distance because it increases sensitivity and allows for measuring smaller distances (for the same signal SNR).

The value of ϵᵣ affects not only how the signal travels through a material, but also affects what happens at the interface between two materials. Consider a signal traveling from a material with relative permittivity ϵᵣ₁ to a material with relative permittivity ϵᵣ₂. Further, for ease of exposition, assume that the signal is traveling perpendicular to the interface, which is the direction of minimum reflection. In this case, the ratio of the reflected power Pᵣ and incident power Pₜ is given by:

$$\frac{{P}_{r}}{{P}_{t}} = {\vert \frac{\sqrt{{ϵ}_{r 1}} - \sqrt{{ϵ}_{r 2}}}{\sqrt{{ϵ}_{r 1}} + \sqrt{{ϵ}_{r 2}}} \vert}^{2} \qquad ( 4 )$$

As can be seen in the equation, larger the difference between the properties of two materials, the more signal power is reflected.

As discussed above, when an RF signal traverses the interface between two materials, it experiences a change in direction. This bending in the signal is called refraction. The relationship of the angle of incidence (θᵢ) and the angle of refraction (θₜ) can be approximated by the following equation (for exact equation, see [25]):

Re(\sqrt{ϵᵣ₁})sin θᵢ=Re(\sqrt{ϵᵣ₂})sin θₜ (5)

where Re(.) denotes the real part of a complex number. The RF signals therefore experience significant bending at skin-fat, fat-muscle and air-skin interfaces. This is the key challenge for localization of a device implanted inside the body. Based on this refraction, this means that it does not matter how the signal arrives in air, it enters the body almost along the direction of the normal on the surface. Since EM wave paths are reversible, this result means that all RF waves that exit the body must arrive at the skin-air interface almost perpendicular to the body surface. RF waves that try to exit the body with an angle far from the normal are not allowed to do so, and are reflected back inside the body. This observation is used in the localization approach.

Principles of Positioning

Conventional state-of-the-art RF localization systems generally operate in two steps. In the first step, they use the phase of the wireless channel between the transmitter and the receiver to measure the angle-of-arrival of the signal or distance between the transmitter and the receiver. In the second step, they assume the path traveled by the signal is straight, and apply basic geometry to locate the transmitter. For in-body RF signals, both these steps are bound to fail if applied as is.

First, let us consider the phase of the wireless channel. As shown in Eq. 1, the phase in air or vacuum, ϕ, is given by:

$$φ = - 2 π f \frac{d}{c}$$

mod 2π. Here, j is the frequency of the signal and c is the speed of EM waves in vacuum. Hence, the phase of the wireless channel linearly depends on the distance traveled by the signal. In contrast, the phase accumulated by the signal in a biomaterial is scaled by α=Re(\sqrt{ϵᵣ}), where ϵᵣ is the electrical permittivity of the material. Thus, when the signal traverses the body, its phase is:

$$φ = - 2 π \frac{f}{c} ( {\sum }_{i} {α}_{i} {d}_{i} ) mod2 π , \qquad ( 6 )$$

where the sum is over the various materials traversed by the signal (air, skin, fat, muscle, etc.), αᵢ is the real square-root of the electrical permittivity of material i, and dᵢ is the distance traveled in that material. This means that when the signal traverses multiple materials, the phase is no longer a simple function of the distance between the transmitter and the receiver.

Second, even if we could measure the distance traversed or angle-of-arrival of the signal, how does one map it to a location? The signal experiences refraction (bending) at the interface between different material (e.g., the interface between air and skin). Thus, the assumption that the signal travels between two points along straight lines no longer holds. As a result, the geometric model of intersecting distances or angles from multiple viewpoints doesn't apply.

Third, we need to take into account that our signal travels the entire distance at different frequencies. The signal from the transmitter to the backscatter device consists of two frequencies (f₁ and f₂), while the signal measured at the receiver after being backscattered is at f₁+f₂, 2f₁+f₂, and other frequency combinations. Together, these three factors are the main challenges facing in-body localization.

Before introducing a specific positioning algorithm used in one or more embodiments, we discuss a few key insights on which the algorithm is built. The first insight is that RF signals exit the body from a small region on the surface. Above, we made the observation that it does not matter how the signal arrives from air, it enters the body only close to the direction of the normal on the surface. Since RF propagation is reversible, this means that it is also not possible for the RF signal to escape from the body through all possible directions. In fact, it can escape only from a small region around the normal on the surface, as shown in FIG. 6. The reason is the property of refraction. Specifically, when an RF signal travels from a high permittivity material (like human body) to a low permittivity material (like air), it bends away from the direction perpendicular to the interface between the materials. Substituting the electrical permittivity values for body tissues in Eq. 5 shows that the cone in FIG. 6 is about 8°. In-body signals that arrive more than 8-10° away from the normal on the surface typically reflect internally and do not escape to the air.

As a corollary of the first point, in-body multipath either does not exist or is very weak compared to the direct path. Any signal that is reflected back into the body has to traverse multiple cm of human tissue and face multiple reflections before it can escape the human body. Because of the exponential attenuation caused by human tissue, this signal will be very low power compared to the direct path emanating out of the body. This is quite in contrast to large scale in-air localization systems where the line-of-sight path can be much weaker than multipath because of obstructions.

Finally, the human body has multiple layers of tissues interleaved with each other. For example, skin and muscle are alike in electrical properties but are separated by fat which is closer to air.

Further, the same material can appear in multiple layers (e.g., air-skin-fat-muscle-fat-msucle). This complex layering structure makes it challenging to model refraction at various interfaces. However, for parallel layers, order and interleaving can be changed with no impact on the total phase of the signal. (Note that reordering of layers does affect the amplitude due to more reflections.) Since human tissues tend to be layered on top of each other, the assumption of parallelism is a reasonable approximation. This observation implies that the multiple layers of the human body can be rearranged for modeling and approximated to be grouped in two major layers: one layer comprising oil based tissues (like fat) and another layer comprising water based tissues (like skin and muscle).

Positioning Algorithm

A particular localization algorithm used in one or more embodiments has two steps. First, it estimates the distances traveled by the signal as if it were traveling in air. We call such values the effective-in-air distances. Second, it models signal paths with linear splines (piecewise linear segments). The length of each segment refers to the stretch of the path in a particular material (air, fat, muscles). It then solves an optimization problem that maps the effective distances to the correct splines that match the actual paths traveled by the signal. (For simplicity, all phase equations are expressed ignoring the initial difference in oscillator phase between transmitter and receiver which can be measured during the calibration phase.)

Measuring Effective in-Air Distances

Consider a signal traveling from a transmitter to a receiver through L different biomaterials. Assume that it travels distance dᵢ in biomaterial i, with phase scaling factor αᵢ=Re(\sqrt{ϵᵣᵢ}). We define effective in-air distance, d_{eff}, traveled by the signal as:

$${d}_{eff} = {\sum }_{i = 0}^{N - 1} {α}_{i} {d}_{i} \qquad ( 7 )$$

Combining Eq. 7 with Eq. 6, the phase, ϕ, of the signal observed by the receiver is:

$$φ = - \frac{2 π {fd}_{eff}}{c} mod 2 π . \qquad ( 8 )$$

Thus, an alternative definition for the effective in air distance is that, if traveled in air, it would result in the received phase.

So, how do we compute the effective distances? Recall that the system has two transmit antennas that transmit f₁ and f₂, respectively, and a number of receive antennas. Let d₁ and d₂ be the effective distances from the two transmitters to the backscatter device, and dᵣ the effective distance from the backscatter device to receiver r. The transmitters are transmitting frequencies f₁ and f₂, while the receivers receive the non-linear mixing of these two signals at frequencies f₁+f₂, 2f₁−f₂, and other linear combinations. Let us consider the phase of f₁+f₂ measured at receive antenna r, which can be given by:

$${φ}_{i} = - \frac{2 π}{c} ( {f}_{1} {d}_{1} + {f}_{2} {d}_{2} + ( {f}_{1} + {f}_{2} ) {d}_{r} ) mod 2 π \qquad ( 9 )$$

This phase equation is a combination of three components. The first two components correspond to the phase of the signal from the transmit antenna to the device. They combine based on the particular non-linear component of the signal that we receive. Since, we are considering just the non linear component f₁+f₂, which is just the sum of the frequencies, the corresponding phases also add up. For example, if we were to consider the frequency component 2f₁−f₂, then f₁d₁+f₂d₂ in Eq. 9 would be replaced by 2f₁d₁−f₂d₂. Eq. 9 gives us one equation in terms of three unknowns, d₁, d₂, and dᵣ. We need more equations to solve for these unknowns. Note, now, that the non-linearity generates various frequency mixes, which provide additional equations. For example, we can write a similar equation for 2 f₁−f₂. The phase, ψᵢ, measured at this frequency is given by:

$${ψ}_{i} = - \frac{2 π}{c} ( 2 {f}_{1} {d}_{1} - {f}_{2} {d}_{2} + ( 2 {f}_{1} - {f}_{2} ) {d}_{r} ) mod 2 π \qquad ( 10 )$$

Once again, note that the phase accumulated by the signal combines in the same way as the frequencies.

To simplify Eq. 9 and Eq. 10, we combine them as:

$$\begin{matrix}{φ}_{i} + {ψ}_{i} = - \frac{2 π}{c} 3 {f}_{1} ( {d}_{1} + {d}_{r} ) mod 2 π & ( 11 ) \\ 2 {φ}_{i} - {ψ}_{i} = - \frac{2 π}{c} 3 {f}_{2} ( {d}_{2} + {d}_{r} ) mod 2 π & \end{matrix}$$

Thus, we get equations expressed as summed distances from each of the transmitters to the receivers. At this point, we cannot use more harmonics to solve for individual distances since they will just yield equations that are linearly dependent on these two harmonics. However, we can use another receiver r′ to get two additional equations that are functions of d₁, d₂ and d_{r′}. Thus, given at least two receive antennas, these four equations can be solved to obtain d₁, d₂, dᵣ and d_{r′}. More antennas can be used to improve accuracy of distance estimates. We note that all phase equations are mod 2π. To resolve ambiguity due to the phase wrapping around, the system optionally, uses a small frequency band around each of the transmitted frequencies—i.e., instead of just transmitting f₁ and f₂, the system sweeps through its transmission in a small band of 10 MHz around f₁ and f₂.

Mapping Effective Distance to Actual Location

Now that we have the effective in-air distances between the in-body backscatter device and the transmit and receive antennas outside the body, we want to map those effective distances to the actual physical location of the backscatter device. At this stage we can drop the distinction between the transmit and receive antennas and treat all effective distances in the same way.

As we said before, the in-air effective distance does not translate into physical distances directly. Furthermore, since the signals do not travel in a straight line, the distances cannot simply be intersected from the different transmitters and receivers to get the right location. To solve this problem, we model signal propagation inside the human body as linear splines (instead of a straight line). Thus, propagation in each layer is linear, but across layers, it can change directions.

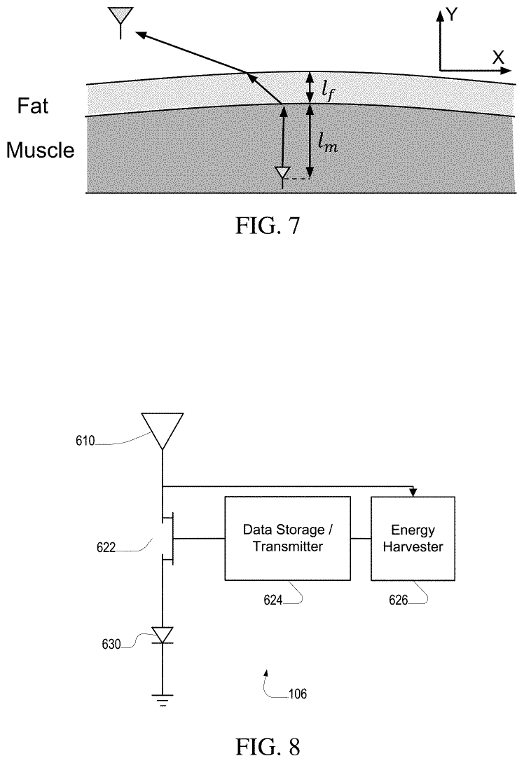

As discussed above, human tissues can be classified as either oil (like fat) or water based (like muscle). Furthermore, different layers can be rearranged such that the muscle-based tissues occur together and the fat-based tissues occur together. Thus, we model the human body as a two-layer system, as shown in FIG. 7. For ease of exposition and visualization, we discuss the algorithm in the 2D XY plane. An extension to 3D is straightforward. The in-body backscatter module is located at X, where X is a tuple of its (x, y) coordinates. The implant is covered by a layer of muscle with depth lₘ (relative permittivity ϵᵣₘ). Then, there is a layer of fat, which has depth l_{f} (relative permittivity ϵ_{rf}). Thus, our model, [symbol] has three latent variables (X, lₘ, l_{f}). In addition to these latent variables, the model has fixed parameters, Θ: the position of each antenna, Xᵢ for i=1, . . . , N, and the permittivity of biomaterials ϵ_{rf} and ϵᵣₘ. The observations made by the model are the effective distance measurements, dᵢ, from the implant to each of the antennas. Then, the goal of the model is to estimate the hidden variable (X, lₘ, l_{f}) given a set of observations.

Next, how does the model constrain the structure of the splines for each path? Let us consider the effective in-air distance dᵢ measured at the i^{th} antenna. In our model, the effective in-air distance is modeled by a spline comprised of 3 different segments: an in-air segment of length dₐⁱ, in-fat segment of length d_{f}ⁱ. and in-muscle segment, dₘⁱ. Together, when these physical distances are scaled by their respective scaling factors and summed together, they should yield the effective-in-air distance dᵢ. The estimation of the individual segments of these splines is governed by two sets of constraints:
Refraction Constraints: Let us say the angle of incidence inside fat, muscle and air is θ_{f}ⁱ, θₘⁱ, θₐⁱ respectively. Then:

Re(\sqrt{ϵᵣₐ})sin θₐⁱ=Re(\sqrt{δ_{rf}})sin θ_{f}ⁱ

Re(\sqrt{ϵᵣₘ})sin θₘⁱ=Re(\sqrt{ϵ_{rf}})sin θ_{f}ⁱ (12)
Geometric Constraints: If (Xᵢ−X)₁ denotes the horizontal dimension of the difference between two positions, then:

$$\begin{matrix}{d}_{a}^{i} = \frac{{l}_{a}}{cos {θ}_{a}^{i}} , {d}_{f}^{i} = \frac{{l}_{f}}{cos {θ}_{f}^{i}} , {d}_{m}^{i} = \frac{{l}_{m}}{cos {θ}_{m}^{i}} & ( 13 ) \\ {d}_{a}^{i} sin {θ}_{a}^{i} + {d}_{f}^{i} sin {θ}_{f}^{i} + {d}_{m}^{i} sin {θ}_{m}^{i} = {( {X}_{i} - X )}_{1} & \end{matrix}$$

where lₐ is the depth of air which is equal to the total distance along the vertical dimension minus the depth of muscle and fat combined.

We now have a system with 6 variables (dₐⁱ, dₘⁱ, d_{f}ⁱ, θₘⁱ, θ_{f}ⁱ, θₐⁱ) and 6 independent equations (Eq. 12 and 13). This is solvable numerically using ray tracing methods. Finally, dₐⁱ, d_{f}ⁱ and dₘⁱ, thus obtained are functions of the latent variables (X, lₘ, r_{f}). Thus, we denote the length of the segments of the spline corresponding to antenna i in air, fat and muscle by dₐⁱ(X, lₘ, l_{f}), d_{f}ⁱ(X, lₘ, l_{f}), dₘⁱ(X, lₘ, l_{f}) respectively.

Now that we modeled the individual segments of the splines as functions of the latent variables in the model, we want to leverage the observed effective in-air distances to estimate the latent variables. Specifically, for each antenna, we minimize the L2-norm of the observed effective in-air distances (dᵢ) and the distance obtained by the scaled sum of the spline segments (dₐⁱ(.)+α_{f}d_{f}ⁱ(.)+αₘdₘⁱ(.)). As before, α=Re(\sqrt{ϵᵣ}). Thus, combining distance measurements from multiple antennas, our optimization function can be written as:

$$\hat{X} , \hat{{l}_{m}} , \hat{{l}_{f}} = arg {min}_{X , {l}_{m} , {l}_{f}} {\sum }_{i = 1}^{N} {‖ {d}_{a}^{i} ( \cdot ) + {α}_{f} {d}_{f}^{i} ( \cdot ) + {α}_{m} {d}_{m}^{i} ( \cdot ) - {d}_{i} ‖}^{2} \qquad ( 14 )$$

where \hat{X}, \hat{l}ₘ, \hat{l}_{f} are the optimal values of the latent variables. This optimization problem is convex in each of the hidden variables (X, lₐ, l_{f}) for ϵᵣ value ranges of human tissues. Furthermore, it has one local maximum. It can be framed as a standard convex optimization problem and solved using convex optimization techniques. By doing so, this optimization can accurately estimate the position of the device by modeling the spline structure.

Alternatives and Implementations

Referring to FIG. 8, an embodiment of the backscatter module 106 for use for both localization and data transmission from the module includes an antenna 610, which acquires the signals 104a from the transceiver 102. In an experimental embodiment, a PC30 dipole antenna from Taoglas [30] was used. However, this antenna is 7.5 cm long and its gain is around 0 dB in-air for the band of interest. Smaller antennas the size ([11, 23, 20]) of a grain of rice have been used in in-body Radio Frequency Identification (RFID) device, and such smaller antennas may be used in practical implementations of the backscatter module.

The non-linear circuitry that causes generation of non-linear combinations of the input frequency components can comprise a diode 630. For example, a Schottky detector diode from Skyworks Solutions [29] is used. Other passive or potentially active non-linear components may be used to induce the non-linear behavior that causes emission of the harmonics of the input frequencies.

In embodiments that provide an output data channel from the backscatter module, a data storage and transmitter component 624 may include circuitry for serializing data stored in or accessible to the backscatter module. Such a serialized data stream may be used switch a transistor 622 so that the generation of the non-linear components is gated in time. In embodiments in which there is no permanent or long-term power source, an energy harvester 626 may be coupled to the antenna 610 to convert received RF energy to power useable by the data storage and transmission component. Not shown in FIG. 8 is optional receiver circuitry that may be used to extract information encoded in the received signals for use in the backscatter module.

Regarding the transceiver, as introduced above, two transmit antennas 104a and corresponding transmit chains may be used to keep the transmit frequencies separate and avoiding generating harmonic components before emission in to the air. However, with suitable circuitry (e.g., power amplifier linearization etc.) a single transmit chain and transmit antenna may be sufficient. Also as introduced above, whereas multiple receive antennas 104b are useful for localization, if only data communication from the backscatter device is needed, then a single receive antenna may be sufficient. Even in the case of communication only, multiple receive antennas at different locations may nevertheless be useful, for example, to increase the overall signal-to-noise ratio in extracing the coded data in the backscattered signal. Furthermore, more than three receive antennas may be useful to increase the number of transmit-receive antenna pairs, thereby providing more constraints that may be used to determine the location and values of the various latent variables (e.g., layer thicknesses and properties, etc.). Various physical arrangements of the antennas may be used, for example, being arranged in an array placed close to the patient's body.

As introduced above, the backscatter module may be coupled to a variety of in-body devices, such as pacemakers, smart sensors that image the body (e.g., intestine) or measure physical and/or chemical properties of the body. The combined sensor and backscatter module may be relatively permanently affixed in the body (e.g., surgical implantation) or may be transient in the body (e.g., a “smart pill” that is ingested). In some examples, the in-body device is controlled by the received RF signals, for example, being triggered to sense the body when the signal is received and/or to receive control information encoded in the received RF signals. In many embodiments, no batter or long-term energy storage device is incorporated in the in-body device.

Embodiments of the transceiver (e.g., the out-of-body component of the system) may be implemented in software, in hardware, or a combination of software and hardware. Software components may include instructions stored on non-transitory machine-readable media, and these instructions cause processors to perform steps described above. For example, the localization algorithm may be implemented in a software-based module of the system. As another example, the radio transmission and reception may use a software-based radio. Some modules may use hardware (e.g., analog circuit) implementations. For example, the filtering of the originally transmitted frequencies may be performed in hardware component between the antenna and a software based radio such that the received signal is not digitized until after the interfering frequency components are filtered out. Some implementations may used hardware components that include special-purpose digital circuitry to implement modules of the system. For example, such hardware components may include application-specific integrated circuits (ASICs) or field-programmable gate arrays (FPGAs). Embodiments of the backscatter module include passive circuit components, and may further include software based components for data transmission and access to data storage elements in the device.

It is to be understood that the foregoing description is intended to illustrate and not to limit the scope defined by the appended claims. Other embodiments are within the scope of the following claims.

What is claimed is:

1. A localization method comprising:
receiving, at each antenna of a plurality of antennas, an emitted signal from a passive device located in a subject's body, each antenna of the plurality of antennas providing a respective received signal of a plurality of received signals, wherein the emitted signal includes a first set of frequency components and is caused by subjecting the passive device to a transmitted signal including a second set of frequency components not included in the first set of frequency components; and
processing the plurality of received signals to determine a location of the passive device, the processing being based at least in part on the effects of different propagation speeds of the emitted signal in one or more layers of tissue through which the emitted signal passes to reach the plurality of antennas.

2. The method of claim 1 wherein the passive device includes circuitry for forming the first set of frequency components from the second set of frequency components.

3. The method of claim 2 wherein the circuitry is non-linear circuitry.

4. The method of claim 2 wherein the circuitry includes a diode.

5. The method of claim 1 wherein the transmitted signal includes a first frequency component with a first frequency and a second frequency component with a second frequency and the first set of frequency components includes a mixture of the first frequency and the second frequency.

6. The method of claim 1 further comprising processing the received signals to remove components at frequencies of the second set of frequency components.

7. The method of claim 6 wherein processing the received signals to remove components comprises passing antenna signals through analog filters prior to digitization.

8. The method of claim 1 wherein determining the location of the passive device includes determining a first set of distances between the plurality of antennas and the passive device and determining a second set of distances by processing the first set of distances according to the effects of the different propagation speeds of radio frequency signals in the one or more layers of tissue through which the emitted signal passes to reach the plurality of antennas.

9. The method of claim 1 wherein the effects of the different propagation speeds of radio frequency signals in the one or more layers of tissue through which the emitted signal passes to reach the plurality of antennas include refraction and changes in wavelength.

10. The method of claim 1 wherein the location of the passive device is determined, at least in part, using a model of human tissue.

11. The method of 10 wherein the model defines the one or more layers of tissue as including an oil-based tissue layer and a water-based tissue layer.

12. An in-body device comprising:
an antenna; and
a non-linear circuit coupled to the antenna;
wherein the combination of the antenna and the non-linear circuit is configured to, when excited by a radio frequency signal including signal components at a first set of two of more frequencies, emit a radio frequency signal including signal components at a second set of frequencies that is distinct from the first set of frequencies.

13. The in-body device of claim 12 wherein the non-linear circuit comprises a diode.

14. The in-body device of claim 13 wherein the non-linear circuit comprises a Schottky detector diode.

15. The in-body device of claim 12 wherein the non-linear circuit is a passive circuit.

16. The in-body device of claim 12 further comprising circuitry configured to modulate the emitted radio frequency signal.

17. The in-body device of claim 16 wherein the circuitry configured to module the emitted radio frequency signal comprises a modulating element coupled to the antenna and the non-linear circuit.

18. The in-body device of claim 17 wherein the modulating element comprises a transistor.

19. The in-body device of claim 17 wherein circuitry configured to modulate the emitted radio frequency signal comprises transmission circuitry for receiving data and outputting a control signal from controlling the modulating element.

20. The in-body device of claim 16 further comprising a sensor configured to acquire sensor data in the body and wherein the device is configured to modulate the emitted radio frequency signal according to the acquired sensor data.

21. The in-body device of claim 20 wherein the sensor comprises at least one of a camera, and electrical sensor, and a biochemical sensor.

22. The in-body device of claim 12 further comprising an energy harvesting component coupled to the antenna configured to convert received radio-frequency energy to power for operating circuitry of the in-body device.

23. A kit comprising an external device configured to
receive, at each antenna of a plurality of antennas, an emitted signal from a passive device located in a subject's body, each antenna of the plurality of antennas providing a respective received signal of a plurality of received signals, wherein the emitted signal includes a first set of frequency components and is caused by subjecting the passive device to a transmitted signal including a second set of frequency components not included in the first set of frequency components; and
process the plurality of received signals to determine a location of the passive device, the processing being based at least in part on the effects of different propagation speeds of the emitted signal in one or more layers of tissue through which the emitted signal passes to reach the plurality of antennas.

24. The kit of claim 23 further comprising a passive device for introduction into a subject, the passive device configured to emit the emitted signal as a result of being subjected to the transmitted signal.
